(19) United States
(12) Patent Application Publication
(10) Pub. No.: US 2025/0331808 A1
(43) Pub. Date: Oct. 30, 2025

(54) MATERNAL AND FETAL MONITORING SYSTEMS AND METHODS

(71) Applicant: GE Precision Healthcare LLC, Waukesha, WI (US)

(72) Inventors: Shrihari Viswanath, Bengaluru (IN); Nagendran Raja, Bengaluru (IN); Rajendra Naik, Bengaluru (IN)

(73) Assignee: GE Precision Healthcare LLC, Waukesha, WI (US)

(21) Appl. No.: 18/644,742

(22) Filed: Apr. 24, 2024

(51) Int. Cl. A61B 8/08; A61B 8/00; A61B 8/02

(52) U.S. Cl. CPC A61B 8/0866; A61B 8/02; A61B 8/4236; A61B 8/4477; A61B 8/5276; A61B 8/54; A61B 8/56

(57) ABSTRACT

A maternal and fetal monitoring system comprises a first ultrasound transducer configured to be positioned on a maternal patient abdomen to acquire fetal ultrasound measurements of a fetus, the first ultrasound transducer housed in a first housing; a maternal measurement patch configured to be secured on the maternal abdomen and to obtain UA physiological measurements indicative of uterine activity (UA) of the maternal patient; a connection cable connecting the maternal measurement patch to the first housing and configured to transmit the UA physiological measurements from the maternal measurement patch; and a controller configured to determine fetal heart rate (fHR) values for the fetus based on the fetal ultrasound measurements and to determine UA values for the maternal patient based on the UA physiological measurements.

### BACKGROUND

The present disclosure generally relates maternal and fetal monitoring, and specifically to a device and method for monitoring maternal and fetal heart rates and maternal uterine activity.

Prior to the onset of labor, a pregnant patient prefers to be ambulatory. In other words, the pregnant patient prefers to be able to move about freely, whether in the patient's own home or within the hospital. However, a pregnant patient who is likely to begin labor soon has reduced ambulatory ability due to the number of sensors that are normally attached to their abdomen to monitor both the onset of labor and the health of the unborn baby.

Sensors are often attached to a pregnant patient during pre-labor and intra labor for monitoring the fetal heart rate (fHR) and uterine activity (i.e. maternal contractions). Additionally, maternal heart rate is another important parameter to monitor maternal health apart from fetal health. Various sensor arrangements and monitoring systems are available for tracking fetal heart rate (fHR), uterine activity (UA), and maternal heart rate (mHR). For example, systems are known that are configured to detect a fetal electrocardiogram (FECG) and/or fHR without making physical contact with the fetus. For example, some monitoring systems use electrodes configured to be placed on the mother's skin about the abdomen to detect electro physiological signals. The maternal electrocardiogram (MECG) and/or mHR can also be detected by electrodes. Uterine activity can also be determined from electrophysiological signals. One example of such a system is Novii wireless patch system available from GE Healthcare.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the disclosure, a maternal and fetal monitoring system comprises a first ultrasound transducer configured to be positioned on a maternal patient abdomen to acquire fetal ultrasound measurements of a fetal patient, the first ultrasound transducer housed in a first housing; a maternal measurement patch configured to be secured on the maternal abdomen and to obtain UA physiological measurements indicative of uterine activity (UA) of the maternal patient; a connection cable connecting the maternal measurement patch to the first housing and configured to transmit the UA physiological measurements from the maternal measurement patch; and a controller configured to determine fetal heart rate (fHR) values for the fetal patient based on the fetal ultrasound measurements and to determine UA values for the maternal patient based on the UA physiological measurements.

In one embodiment, the maternal measurement patch is configured to be secured at or near a fundus of the maternal abdomen.

In another embodiment, the connection cable is configured to enable placement of the first ultrasound transducer level with or below the umbilicus while the maternal measurement patch is secured at or near the fundus of the maternal abdomen.

In another embodiment, the first ultrasound transducer is configured to be secured level with or below the umbilicus of the maternal abdomen and the maternal measurement patch is configured to be secured above the umbilicus of the maternal abdomen.

In another embodiment, the maternal measurement patch includes an adhesive and is configured to be secured to the maternal abdomen by adhering thereto.

In another embodiment, the maternal measurement patch is a single-use disposable device.

In another embodiment, the connection cable includes a connection end configured to removably connect to a connection port on the first housing.

In another embodiment, the UA physiological measurements indicative of UA of the maternal patient include ultrasound measurements, light transmission measurements, force measurements, electrical potentials, or any combination thereof.

In another embodiment, the maternal measurement patch includes a second ultrasound transducer configured to acquire UA ultrasound measurements indicative of the UA of the maternal patient, wherein the controller is configured to determine the UA value based on the UA ultrasound measurements.

In another embodiment, the second ultrasound transducer emits and measures a different ultrasound frequency than the first ultrasound transducer.

In another embodiment, the controller is configured to control operation of the second ultrasound transducer to acquire the UA ultrasound measurements when the first ultrasound transducer is not operating to acquire the fetal ultrasound measurements.

In another embodiment, the maternal measurement patch includes a light transmission measurement device configured to acquire light transmission measurements indicative of the UA of the maternal patient, wherein the controller is configured to determine the UA value based on the light transmission measurements.

In another embodiment, the maternal measurement patch is further configured to acquire light transmission measurements indicative of maternal heart rate (mHR) values, and wherein the controller is further configured to determine a heartbeat coincidence based on the fHR values and the mHR values.

In another embodiment, the maternal measurement patch includes a force gauge sensor configured to acquire force measurements indicative of the UA of the maternal patient. Optionally, the force gauge is a MEMS-based strain gauge.

In another embodiment, the maternal measurement patch includes a plurality of EMG sensors configured to acquire electrical potentials indicative of the UA of the maternal patient.

In another embodiment, the controller is housed in the first housing and further comprising a wireless transmitter in the first housing and configured to transmit at least the UA value and the fHR value to a receiving device.

In another embodiment, the maternal and fetal monitoring system further comprises a wireless transmitter in the first housing and configured to transmit the fetal ultrasound measurements and the UA physiological measurements to an external patient monitor, wherein the controller is in the external patient monitor.

In another embodiment, the maternal measurement patch is further configured to acquire measurements indicative of maternal heart rate (mHR) values, and wherein the controller is further configured to determine a heartbeat coincidence based on the fHR values and the mHR values.

In another embodiment, the maternal and fetal monitoring system further comprises a maternal heart rate monitor configured to be worn by the maternal patient and configured to transmit maternal heart rate (mHR) values, wherein the controller is further configured to determine a heartbeat coincidence based on the fHR values and the mHR values.

In another embodiment, the controller is configured to receive maternal heart rate (mHR) values from a maternal heart rate monitor configured to be worn by the maternal patient and configured to transmit the mHR values, wherein the controller is further configured to determine a heartbeat coincidence based on the fHR values and the mHR values. Optionally, the maternal heart rate monitor is a wrist-worn heart rate monitor configured to be worn on the maternal patient's wrist.

In another embodiment, the maternal and fetal monitoring system further comprises an accelerometer configured to measure motion of the maternal patient, wherein the controller is further configured to receive maternal motion data from the accelerometer and to use the maternal motion data to remove artifact from the UA physiological measurements and/or from the fHR values.

In another aspect of the disclosure, a method of maternal and fetal monitoring comprises operating a first ultrasound transducer configured to be positioned on a maternal patient abdomen to acquire fetal ultrasound measurements of a fetal patient; determining fetal heart rate (fHR) values for the fetal patient based on the fetal ultrasound measurements; when the first ultrasound transducer is not operating to acquire the fetal ultrasound measurements, obtaining UA physiological measurements indicative of uterine activity (UA) of the maternal patient via a maternal measurement patch configured to be secured on or near the fundus of the maternal abdomen; and determining a UA value for the maternal patient based on the UA physiological measurements.

In another embodiment, the UA physiological measurements indicative of UA of the maternal patient include ultrasound measurements, light transmission measurements, force measurements, electrical potentials, or any combination thereof.

In another embodiment, the method of maternal and fetal monitoring further comprises when the first ultrasound transducer is not operating to acquire the fetal ultrasound measurements, obtaining physiological measurements indicative of maternal heart rate (mHR) via the maternal measurement patch, and wherein the controller is further configured to determine mHR values based on the physiological measurements and to determine a heartbeat coincidence based on the fHR values and the mHR values.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 depicts an exemplary maternal and fetal monitoring system positioned on a maternal patient's abdomen according to one embodiment of the present disclosure.

FIG. 2 depicts an exemplary embodiment of a maternal and fetal monitoring system according to the present disclosure.

FIG. 3 is an environmental view including an exemplary embodiment of a maternal and fetal monitoring system according to the present disclosure.

FIG. 4 is a schematic diagram of an exemplary embodiment of a maternal fetal monitor according to the present disclosure.

FIG. 5 is a schematic diagram of an exemplary embodiment of a maternal fetal monitor according to the present disclosure.

FIG. 6 is a schematic diagram of an exemplary embodiment of a maternal fetal monitor according to the present disclosure.

FIG. 7 is a schematic diagram of an exemplary embodiment of a maternal fetal monitor according to the present disclosure.

FIG. 8 shows an exemplary embodiment of the maternal and fetal monitoring system's power control according to the present disclosure.

FIG. 9 shows an exemplary embodiment of a maternal measurement patch according to the present disclosure.

FIGS. 10A and 10B show an exemplary embodiment of a connection end and a connection port according to the present disclosure.

FIGS. 11-12 show exemplary embodiments of a method of maternal and fetal monitoring according to the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

As used herein, the terms controller or module may refer to, be part of, or include an application-specific integrated circuit (ASIC), an electronic circuit, a combinational logic circuit, a field programmable gate array (FPGA), a processor (shared, dedicated, or group) that executes code, or other suitable components that provide the described functionality, or a combination of some or all of the above, such as in a system-on-chip. The terms controller or module may include memory (shared, dedicated, or group) that stores code executed by the processor. The term code, as used herein, may include software, firmware, and/or microcode, and may refer to programs, routines, functions, classes, and/or objects. The term shared, as used above, means that some or all code from multiple modules may be executed using a single (shared) processor. In addition, some or all code to be executed by multiple different processors may be stored by a single (shared) memory. The term group, as used above, means that some or all code comprising part of a single controller or module may be executed using a group of processors. Likewise, some or all code comprising a single controller or module may be stored using a group of memories.

Aspects of the disclosure are described herein in terms of functional and/or logical block components and various processing steps. It should be appreciated that such block components may be realized by any number of hardware, software, and/or firmware components configured to perform the specified functions. For example, an embodiment may employ various integrated circuit components, e.g., memory elements, digital signal processing elements, logic elements, look-up tables, or the like, which may carry out a variety of functions under the control of one or more processors or other control devices. In addition, those skilled in the art will appreciate that the present invention may be practiced in conjunction with any number of medical devices, including any number of different physiological data acquisition devices, and that the system described herein is merely one example application. The connecting lines shown in the various figures contained herein are intended to represent example functional relationships and/or physical couplings between the various elements. It should be noted that many alternative or additional functional relationships or physical connections may be present in a practical embodiment.

The inventors have recognized that current monitoring systems configured to monitor fetal cardiac activity along with maternal cardiac activity and uterine activity are sometimes unreliable for monitoring fetal cardiac activity. It can be challenging to separate the fetal cardiac activity within the electrophysiological signals from signals resulting from the maternal cardiac activity and uterine activity and the noise and other artifacts contained therein. Additionally, maternal adipose tissue and protective layers around the fetus, including the vernix caseosa, can block or impede the biopotentials from the fetal heart such that the fetal ECG is not reliably obtained. Biopotential based determinations of fetal heart rate, maternal heart rate, and uterine activity suffer from other challenges as well. The complex signal processing noted above can take time, resulting in a delay in the reporting of the monitored results, such processing exemplarily taking seconds to complete. However, effective maternal and fetal monitoring requires measurement of both maternal and fetal cardiac activity, and in some systems, it is desirable for the fHR and the UA to be measured simultaneously to interpret and understand the state of oxygenation of the fetus.

In view of the foregoing problems with existing integrated fetal and maternal monitoring systems, the inventors have endeavored to build an integrated maternal and fetal monitoring system that provides a dedicated measurement sensor for fetal cardiac activity, yet provides centralized processing, signal analysis, and data transmission which allows coordinated measurement control and effective signal processing. The disclosed system includes an ultrasound transducer in a housing configured to be positioned on a maternal abdomen and to acquire signals for determining the fetal heart rate (fHR) values. The system further includes a maternal measurement patch configured to be secured to the maternal abdomen and to obtain UA physiological measurements indicative of uterine activity (UA) of the maternal patient. In one embodiment, the patch is connected to the housing of the ultrasound transducer by a connection cable configured to transmit the UA physiological measurements. In another embodiment, the patch is communicatively connected to the ultrasound transducer by short range wireless communication. A controller is configured to determine the fHR values based on the ultrasound physiological measurements and UA values based on the UA physiological measurements.

The fHR sensor is separate from the measurement sensor(s) for uterine activity and for maternal cardiac activity. In some embodiments, the maternal measurement patch is configured to acquire measurements indicative of maternal heart rate (mHR) values (in addition to the UA values). In other embodiments, the controller is configured to receive maternal heart rate (mHR) values from a maternal heart rate monitor configured to be worn by the maternal patient and configured to transmit maternal heart rate (mHR) values, such as from a fitness monitor, a smartwatch, or other type of wrist-word heart rate monitor worn by the maternal patient. The controller is further configured to determine a heartbeat coincidence based on the fHR values and the mHR values.

The maternal measurement patch is configured to be secured at or near a fundus of the maternal abdomen, such as including an adhesive such that it is configured to be secured to the maternal abdomen by adhering thereto. In one embodiment, the maternal measurement patch is a single-use disposable device configured to adhere to the fundus area on the maternal abdomen. The connection cable is configured to enable placement of the first ultrasound transducer level with or below the umbilicus while the maternal measurement patch is secured at or near the fundus. In one embodiment, the housing containing the ultrasound device is held to the maternal abdomen in such a way that it is movable during the monitoring period to adjust the fetal ultrasound measurement area, such as being pressed against the maternal abdomen with a belt. The connection cable is configured to provide slack that that the patch remains adhered at the fundus and stationary while permitting the first ultrasound transducer to be moved.

In another embodiment, the patch is wirelessly connected to the ultrasound transducer, such as short range transmission from the patch to the first transducer. In such an embodiment, the patch includes a wireless transmitter and a battery and is configured to transmit UA physiological measurements as raw data or processed data to the processing system housed with ultrasound transducer, which processes the UA physiological measurements with the ultrasound measurements as described herein.

Fetal heart rate is monitored by a non-invasive ultrasound system, such as using a Doppler ultrasound technique to detect a motion of the beating heart of the fetus. The uterine activity of the pregnant patient is measured by the maternal measurement patch configured to be secured on the maternal abdomen. The UA physiological measurements indicative of UA of the maternal patient may include ultrasound measurements, light transmission measurements, force measurements, electrical potentials, or any combination thereof. For example, the maternal measurement patch may include a second ultrasound transducer configured to acquire UA ultrasound measurements indicative of the UA. Alternatively or additionally, the maternal measurement patch includes a light transmission measurement device configured to acquire light transmission measurements indicative of the UA of the maternal patient. Alternatively or additionally, the maternal measurement patch includes a force gauge sensor configured to acquire force measurements indicative of the UA of the maternal patient. Alternatively or additionally, the maternal measurement patch includes a plurality of EMG sensors configured to acquire electrical potentials indicative of the UA of the maternal patient.

By placing the tocodynamometer or other UA sensor within a patch that can be attached to a maternal patient, such as at the fundus, and by communicatively and adjustably connecting the patch to a housing comprising an ultrasound measurement unit that can be placed elsewhere on the maternal abdomen, the present inventors have recognized that the number and weight of sensors attached to a pre-partum patient's abdomen can be decreased, and thereby the patient's ambulatory ability can be increased. Moreover, by providing a flexible connection cable between the patch and the ultrasound device housing, the system is configured to permit movement and positional adjustment of the ultrasound transducer without disturbing the maternal measurement patch at the fundus, or vice versa. Additionally, by communicatively connecting these sensors a single controller can control both sensors, which allows for coordination of the measurement operations, yielding better power management capabilities, increased artifact elimination, and increased accuracy of the measured fHR.

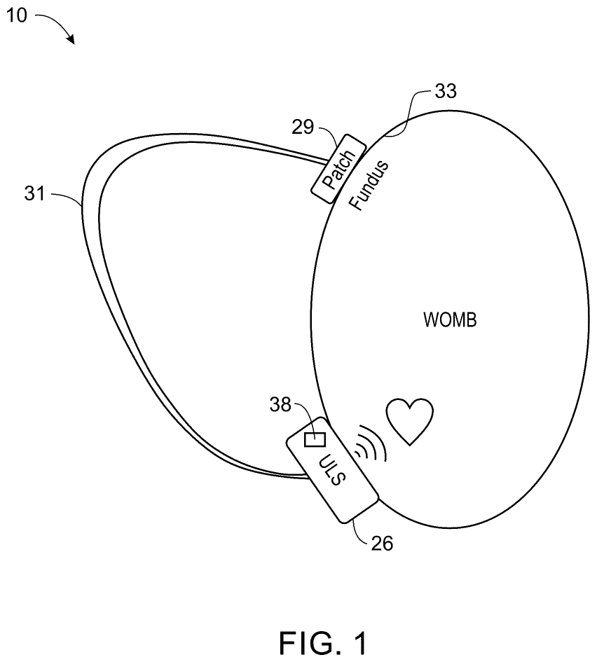

FIG. 1 depicts an exemplary embodiment of maternal fetal monitor 10 for the detection of uterine activity and fetal heart rate. First housing 26 comprises an ultrasound transducer secured on a maternal patient abdomen to acquire fetal ultrasound measurements of a fetal patient. Maternal measurement patch 29 is secured on a maternal patient abdomen to obtain physiological measurements indicative of uterine activity of the maternal patient. A controller 38 located within first housing 26 uses the fetal ultrasound measurements and the uterine activity physiological measurements to determine fetal heart values and uterine activity values, respectively. In some embodiments, maternal measurement patch 29 also monitors maternal heart rate (mHR). Alternatively, in some embodiments maternal heart rate is monitored using a separate device such as a wrist-worn device or finger-worn device.

Maternal measurement patch 29 is removably connected to first housing 26 through flexible connection cable 31. Flexible cable 31 is configured to transmit uterine activity physiological measurements (which may be analog signals or digitized processed or unprocessed signals) from maternal measurement patch 29 to first housing 26. In some embodiments, maternal measurement patch 29 is positioned on the upper portion of the maternal patient's abdomen, near the fundus 33 of the uterus. The fundus 33 is the portion of the uterus which is furthest away from the cervix. In some embodiments, the flexible cable 31 is configured to allow placement of the first housing 26, on the lower portion of the maternal patient's abdomen while the maternal measurement patch 29 is positioned at the fundus 33. Thus, the first housing may be positioned level with or below the umbilicus, near to the location of the fetal patient, in order to detect and track fetal heart rate. Flexible cable 31 is configured to allow various relative positioning of the maternal measurement patch 29 and the first housing 26, and also to allow each element to be moved on the maternal abdomen without disturbing the other. For example, the flexible cable 31 may have a length and flexible construction configured to allow such relative movement. In one embodiment, the flexible cord 31 has a length of at least 15 cm or greater, and in another example may have a length of up to 2 feet.

Maternal measurement patch 29 comprises one or more of several sensors. In some embodiments, patch 29 tracks uterine activity through ultrasound measurements utilizing an ultrasound sensor. In other embodiments, patch 29 tracks uterine activity through light transmission measurements utilizing a light transmission measurement device. In still other embodiments, patch 29 tracks uterine activity through force measurements such as by using a force gauge. In some embodiments, patch 29 tracks uterine activity through electrical potentials utilizing electromyography (EMG) sensors. In alternative embodiments, maternal measurement patch 29 comprises two or more of these sensors combining to track uterine activity.

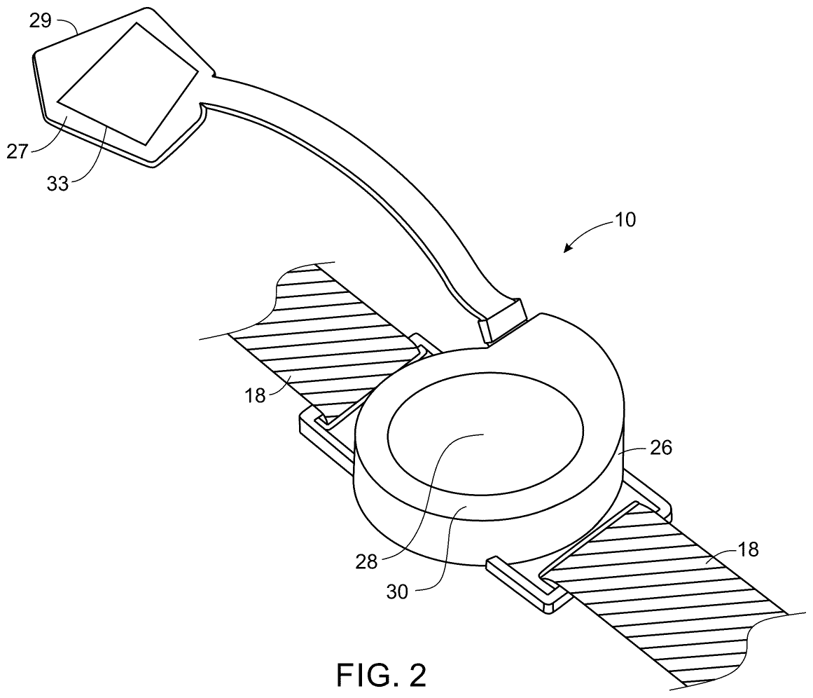

FIG. 2 depicts an exemplary embodiment of a maternal fetal monitor 10 for the detection of uterine activity and fetal heart rate. The figure shows a bottom side 30 of the housing 26 configured to be in contact with the skin of the maternal abdomen and a bottom side 27 of the patch 29 configured to be in contact (such as adhered to) the skin of the maternal abdomen. Sensor 33 may comprise any combination of ultrasound measurements, light transmission measurements, force measurements, or electrical potentials. Sensor 33 is used to detect uterine activity, such as the contraction of the uterus during labor.

Fetal heart rate is monitored using ultrasound transducer 28, located within first housing 26. Ultrasound transducer 28 performs fetal heart rate monitoring based upon Doppler shift in the returned ultrasound signals, as is known in the art. Ultrasound transducer 28 exemplarily includes one or more ultrasound crystals. When the ultrasound crystal(s) receive a suitable excitation signal the ultrasound crystal(s) of the ultrasound transducers 28 produce an acoustic wave therefrom. In an example, the excitation signal delivered to the ultrasound transducer 28 is a 6-volt peak-to-peak sign wave at 1.15 MHz. In a further exemplary embodiment, the 1.15 MHz sign wave produces a brief burst frequency from the ultrasound crystal(s) between 2 kHz-4 kHz. These examples are merely exemplary of the values for the excitation signal and the ultrasound crystal burst frequency and those of ordinary skill in the art will recognize many other values may be used in clinical settings with the excitation signal and the ultrasound burst frequency being coordinated to produce a desirable signal from the ultrasound transducer. In operation, the ultrasound crystals serve as both ultrasound transmitters and ultrasound receivers and the ultrasound transducer 28 is operated to produce the acoustic waveform as described and then operated in a receive mode to receive the returned reflected acoustic signals back at the ultrasound transducer 28. The acoustic signals produced by the ultrasound transducer 36 reflect off of the anatomical structures of the fetal patient, particularly the fetal heart. The movement of the fetal heart causes the doppler shift, which is detected and thus the fetal heart rate is measured. The ultrasound transducer 28 is exemplarily secured to the abdomen of a maternal patient for example by way of an elastomeric strap 18. However, it will be recognized that in other embodiments the housing 26 may be maintained against the skin of the maternal abdomen by other means, such as by a biocompatible adhesive.

Sensor 33 is used to detect and measure the contraction of the uterus. During contractions there might be lack of oxygen for the fetus. During respiratory exchange between the mother and the fetus, blood flow may be compromised by uterine contractions, causing reduced oxygen supply to the fetus. The fetus reacts by trying to pump more blood by beating its heart faster. Other causes might be unnatural and cause harm to the fetus, so the system may be configured to distinguish between acceptable and unacceptable low oxygen by reviewing time-correlated UA physiological measurements and fHR values together to identify whether the contractions are correlated with and/or causing the low oxygen response of the fetus.

Thus, it may be preferable in some configurations to configure the device to measure fetal heart rate and uterine contractions simultaneously, or at least close in time and in the context of the period of a contraction, to understand the state of oxygenation of the fetus.

Additionally, sensor 33 can be used to acquire measurements indicative of maternal heart rate (mHR) values. Specifically, when utilizing electric potentials or light transmission measurements the maternal heart rate values can be calculated from the measurements indicative of maternal heart rate values and thereby monitored. This allows for detection of heartbeat coincidence between fetal heart rate and maternal heart rate. A heartbeat coincidence value indicates a similarity level between the maternal and the fetal heart rate. The heartbeat coincidence may be a binary value, either indicating positive for coincidence or negative for no coincidence, or may be a multi-level value indicating low/high coincidence. A positive or high heart beat coincidence value is an indicator that the measured fetal heart rate may actually be a mismeasurement of the maternal heart rate and not the heart rate of the fetus. By monitoring maternal heart rate, the system can detect when the maternal heart rate and fetal heart rate are so synced up as to indicate they are coming from the same source. An alert can then be generated to a caregiver to reposition the fetal heart rate sensor to ensure it is the fetal heart rate that is being tracked.

FIG. 3 is an environmental view of an exemplary embodiment of a maternal and fetal monitoring system 10 that can be used to simultaneously monitor the fetal patient's heart rate and the maternal patient's uterine activity.

The maternal and fetal monitoring system 10 is exemplarily secured to the abdomen 16 of the maternal patient 14 for example by way of an elastomeric strap 18. However, it will be recognized that in other embodiments a biocompatible adhesive may be used to secure the maternal fetal monitoring system 10 to the patient's abdomen 16.

In embodiments as will be described in further detail herein, the maternal fetal monitoring system 10 may be communicatively connected to an external patient monitor 20. As will be understood by the variety of implementations as described in further detail herein, while all remaining within the scope of the present disclosure, the communicative connection may exemplarily be a wired or a wireless communicative connection. A wireless communicative connection may be a medical body area network (MBAN), and/or may exemplarily use Bluetooth, Bluetooth Low Energy (BLE), ANT or ZigBee communication protocols or other RF communication protocols as may be recognized by a person of ordinary skill in the art. In still further exemplary embodiments, depending upon the configuration of the maternal fetal monitoring system and the data transmitted between the maternal fetal monitoring system 10 and the external patient monitor 20, all or some of the data processing of the physiological information acquired by the maternal fetal monitoring system 10 may be performed locally by a controller within the maternal fetal monitoring system 10, such as by a controller located within first housing 26. The calculated parameters of fetal heart rate, maternal heart rate, uterine activity, or others as described herein may be communicated across the communicative connection to the external patient monitor 20 exemplarily for visual presentation on a graphical display 24 and/or electronic storage of this information on a data network of the hospital or medical facility and exemplarily in an electronic medical record (EMR) of the maternal patient.

Alternatively, a controller 38 located within the external patient monitor 20 may receive the acquired physiological data and process such physiological data in the manners as described herein. In these embodiments, the maternal fetal monitoring system 10 may perform more limited signal processing on the acquired physiological data and provide this “raw” physiological data across the communicative connection to a external patient monitor 20 which applies the signal processing actions and techniques as described herein to calculate the parameters of fetal heart rate, maternal heart rate, uterine activity, and others.

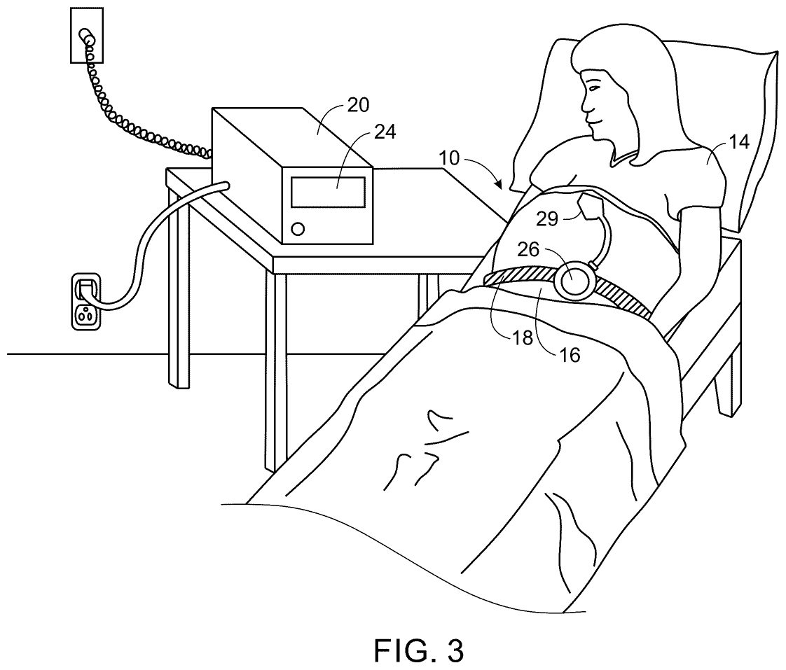

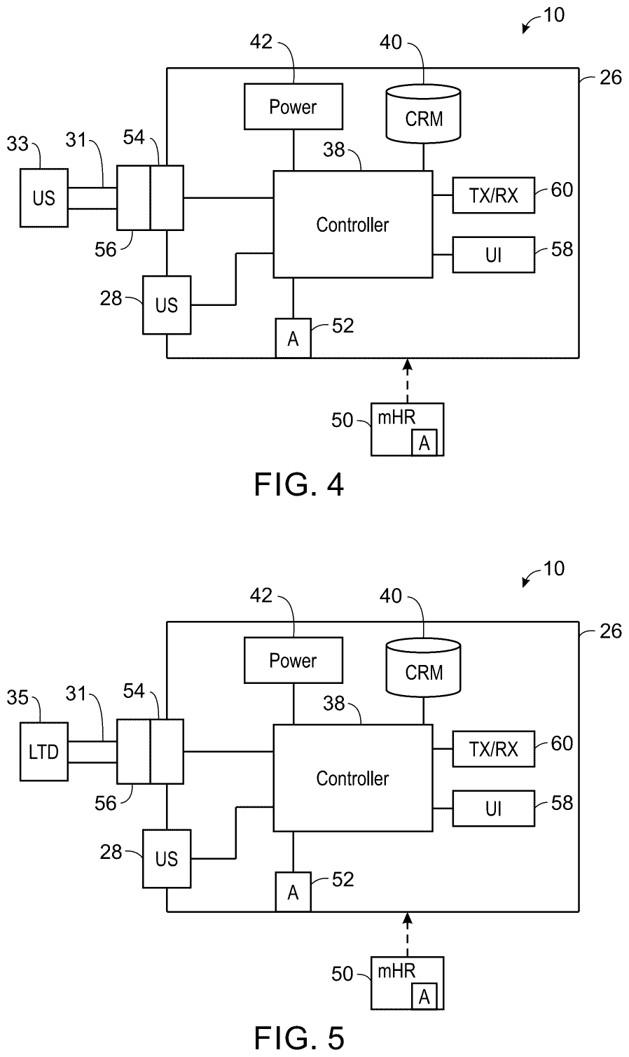

FIG. 4 is a schematic diagram of an exemplary embodiment of a maternal and fetal monitoring system 10. Contained within the first housing 26 is an ultrasound transducer 28. The ultrasound transducer 28 exemplarily includes one or more ultrasound crystals. When the one or more ultrasound crystals receive a suitable excitation signal, the ultrasound crystals of the ultrasound transducer 28 produce an acoustic wave therefrom. In operation, the ultrasound crystals serve as both ultrasound transmitters and ultrasound receivers, and the ultrasound transducer 28 is operated to produce an acoustic wave form as described and then operated in a receive mode to receive the returned reflected acoustic signals back at the ultrasound transducer 28.

The ultrasound transducer 28 is connected to a controller 38. It will be recognized that controller 38 is exemplarily any of a variety of known controller circuits, integrated circuits, micro controllers, microprocessors, and associated circuitry. The controller 38 may exemplarily include a central processing unit (CPU) and integrated memory, although in embodiments the computer readable medium 40 comprising the memory may be a separate component or communicatively connected to the controller within the first housing 26. The controller exemplarily includes a processor that accesses software or firmware in the form of computer readable code stored on non-transient computer readable memory as either integrated memory or external memory. The processor executes the computer readable code as an instruction set to carry out the functions as described herein, including the receipt of input, calculations, and outputs as will be described. The first housing 26 further comprises a power source 42. The power source 42 is exemplarily a battery, such as a rechargeable battery.

The maternal fetal monitor 10 further comprises a second ultrasound transducer 33. Second ultrasound transducer 33 is disposed to be secured to the top half of a maternal patient's abdomen, near to the fundus of the uterus. Second ultrasound transducer 33 is disposed to detect and track the uterine activity of a maternal patient. Second ultrasound transducer 33 exemplarily comprises one or more ultrasound crystals. When the one or more ultrasound crystals receive a suitable excitation signal, the ultrasound crystals of the second ultrasound transducer 33 produce an acoustic wave therefrom. In operation, the ultrasound crystals serve as both ultrasound transmitters and ultrasound receivers, and the second ultrasound transducer 33 is operated to produce an acoustic wave form as described and then operated in a receive mode to receive the returned reflected acoustic signals back at the second ultrasound transducer 33. The acoustic signals produced by second ultrasound transducer 33 reflect off of the anatomical structures of the maternal patient. Second ultrasound transducer 33 utilizes the strength of the returned ultrasonic beam to determine when a muscle contraction has taken place. Interpretation of uterine activity includes the frequency and duration of uterine contractions. Frequency is determined by counting the number of contractions within a predefined period of time and/or minutes between the start of one contraction to the start of the next contraction. Duration is the time (generally indicated in seconds) between the beginning and the end of a contraction. For example, uterine contractions can be indicated as a number (or number range) of contractions within a 10 minute period, and a duration range—e.g., 3 to 5 contractions in the 10-minute window, each lasting 30 to 40 seconds. Second ultrasound transducer 33 is removably connected to first housing 26 through flexible cable 31 connection end 56, and connection port 54. In some embodiments, connection end 56 and connection port 54 are male and female connectors, respectively. In other embodiments, connection end 56 and connection port 54 are female and male connectors, respectively.

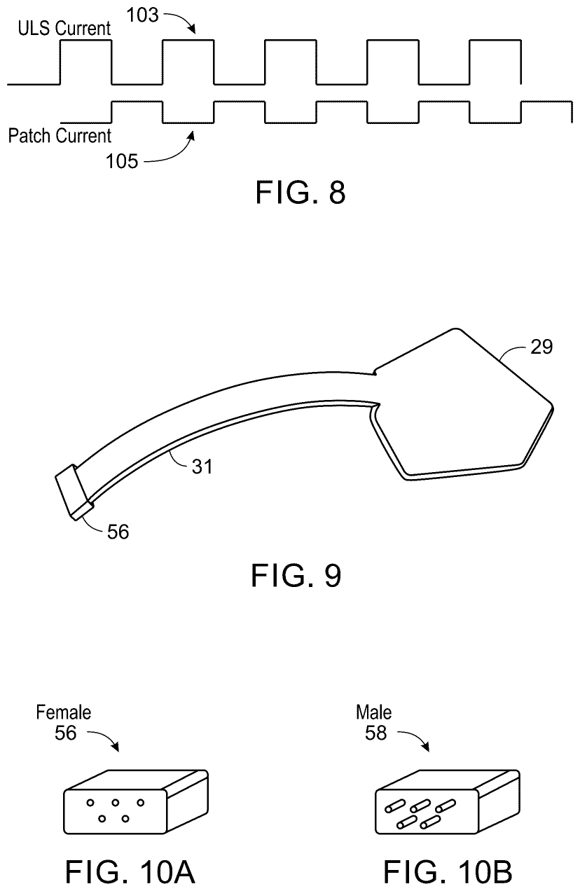

Second ultrasound transducer 33 is controlled by controller 38 through connection end 56, connection port 54, and flexible cable 31. Controller 38 operates second ultrasound transducer 33 such that second ultrasound transducer 33 does not pick up signals from ultrasound transducer 28. Controller 38 utilizes two methods to accomplish this goal. In a first exemplary embodiment, controller 38 operates second ultrasound transducer 33 offset in time from ultrasound transducer 28, such that ultrasound transducer 33 is not transmitting an excitation signal at the same time as ultrasound transducer 28 and is not expecting a return signal at the same time as ultrasound transducer 28. This method also helps accomplish limited power consumption, as demonstrated further herein in FIG. 8. Additionally or alternatively, in another exemplary embodiment, controller 38 operates second ultrasound transducer 33 at a different frequency than ultrasound transducer 28 (that is, emits and measures a different frequency than that emitted and measured by the first transducer 28), such that second ultrasound transducer 33 receives feedback at a different frequency than the feedback received by ultrasound transducer 28. By operating second ultrasound transducer 33 at a different frequency than ultrasound transducer 28, controller 38 ensures that second ultrasound transducer 33 does not pick up emissions (“noise”) from ultrasound transducer 28 that might be misinterpreted as measurements. The second ultrasound transducer 33 also measures at a different (lower) power than the first ultrasound transducer given that it is taking measurements of the uterine wall muscles which are proximal to the location of the transducer.

Also included within first housing 26 is accelerometer 52. Accelerometer 52 may be included within first housing 26 or may be external to first housing 26, for example as part of a wearable maternal heart rate monitor 50, such as a smartwatch, fitness monitor, or other wrist-worn monitoring device. Accelerometer 52 is disposed to detect maternal patient motion data. Accelerometer 52 is communicatively connected to controller 38. Controller 38 utilizes the maternal motion data to determine when there has been movement of the maternal patient. Controller 38 utilizes the information about the movement of the maternal patient to check for and remove artifacts in the fetal heart rate data and/or the from UA physiological measurements. This increases the maternal patient's ambulatory ability as the maternal patient can move without interfering with clear reception of fetal heart rate data.

Also included within first housing 26 is a user interface 58. User interface 58 may be a display screen, a speaker, an LED light bulb, or any other type of interface which can generate an alert to a caregiver who is monitoring a maternal and fetal patient. When controller 38 detects a drop in fetal heart rate such as is commensurate with an indication of fetal distress an alert can be generated through user interface 58 to alert a caregiver of such a condition. Additionally, when, as described above, controller 38 detects a heartbeat coincidence between the maternal patient and the fetal patient an alert can be generated to alert a caregiver to reposition ultrasound transducer 28 to ensure ultrasound transducer 28 is picking up the fetal heart rate and not the maternal heart rate.

Also included within first housing 26 is a transmitter/receiver 60. Transmitter/receiver 60 is configured to transmit raw or filtered physiological data from controller 38 to an external patient monitor via the external patient monitor's receiving device. The transmitter/receiver 60 may be any device known in the art for wirelessly transmitting data between two points. In one embodiment, the transmitter/receiver 60 may be a body area network (BAN) device, such as a medical body area network (MBAN) device, that operates as part of a wireless network of wearable or portable computing devices. Other examples of radio protocols that could be used for this purpose are Bluetooth, Bluetooth Low Energy (BLE), ANT and ZigBee. The external patient monitor may exemplarily display the physiological data for a caregiver's attention or may store the data electronically, such as in an electronic patient record.

Also connected to controller 38, although located outside first housing 26, is maternal heart rate monitor 50. Maternal heart rate monitor 50 is exemplarily a wearable heart rate monitor such as a wrist-worn heart rate monitor or a finger-worn heart rate monitor. The maternal heart rate monitor may be a smartwatch or a fitness monitor, to provide a few examples. Maternal heart rate monitor 50 may exemplarily further comprise an accelerometer configured to detect maternal patient motion data. Maternal heart rate monitor 50 is wirelessly communicatively connected to controller 38 and configured to communicate the maternal heart rate values and/or accelerometer data thereto. The wireless communicative connection may be a medical body area network (MBAN), and/or may exemplarily use Wi-Fi, Bluetooth, or ZigBee communication protocols or other RF communication protocols as may be recognized by a person of ordinary skill in the art.

FIG. 5 is a schematic diagram of an exemplary embodiment of a maternal fetal monitor 10. Contained within the first housing 26 is an ultrasound transducer 28. The ultrasound transducer 28 exemplarily includes one or more ultrasound crystals. When the one or more ultrasound crystals receive a suitable excitation signal, the ultrasound crystals of the ultrasound transducer 28 produce an acoustic wave therefrom. In operation, the ultrasound crystals serve as both ultrasound transmitters and ultrasound receivers, and the ultrasound transducer 28 is operated to produce an acoustic waveform as described and then operated in a receive mode to receive the returned reflected acoustic signals back at the ultrasound transducer 28.

The ultrasound transducer 28 is connected to a controller 38. It will be recognized that controller 38 is exemplarily any of a variety of known controller circuits, integrated circuits, micro controllers, microprocessors, and associated circuitry. The controller 38 may exemplarily include a central processing unit (CPU) and integrated memory, although in embodiments the computer readable medium 40 comprising the memory may be a separate component or communicatively connected to the controller within the first housing 26. The controller exemplarily includes a processor that accesses software or firmware in the form of computer readable code stored on non-transient computer readable memory as either integrated memory or external memory. The processor executes the computer readable code as an instruction set to carry out the functions as described herein, including the receipt of input, calculations, and outputs as will be described. The first housing 26 further comprises a power source 42. The power source 42 is exemplarily a battery.

The maternal fetal monitor 10 further comprises a light transmitter/detector 35. Light transmitter/detector 35 is configured to acquire light transmission measurements indicative of the uterine activity of a paternal patient, such as uterine contractions. Light transmitter/detector 35 is configured to be attached to a maternal patient at the fundus of the uterus. Light transmitter/detector 35 may transmit light in any of the red, green, or IR spectrums. The receiver configured within light transmitter/detector 35 receives light from the transmitter which has been reflected off the anatomical structures of the maternal patient, the muscles around the uterus (particularly those near the fundus). The strength of the reflected light indicates whether or not a uterine contraction is taking place, where a higher concentration of light is reflected when the uterine muscles are contracted than when they are not contracted. Further, the muscles when contracted scatter the light differently for light moving parallel to the muscle and perpendicular to it. By using multiple receivers (photodiodes), this light-scattering behavior can be identified and used to determine the UA value. Uterine contractions may be monitored for both their frequency and their duration. Frequency is determined by counting the minutes between the start of one contraction to the start of the next contraction, which may be measured over multiple contractions and the values may be averaged or otherwise filtered. Duration is the time (generally indicated in seconds) between the beginning and the end of a contraction. For example, uterine contractions can be every 3 minutes, lasting 60 seconds.

Additionally, light transmitter/detector 35 may be used to acquire measurements indicative of maternal heart rate. Light transmitter/detector 35 consists of a light source and an optical receiver. The light source is preferably an infrared LED, a green LED, or a red LED. The receiver detects the changes of the reflected light caused by muscle contractions and by a blood pulse in the arterial vessels of the maternal abdomen. The blood pulse directly corresponds to the maternal heart rate. Thereby, the receiver can directly measure the maternal heart rate by observing changes in the reflected light caused by a blood pulse in the arterial vessels of the maternal abdomen.

In the case where light transmitter/detector 35 is used to monitor maternal heart rate, external maternal heart rate monitor 50 need not be used. Further, when light transmitter/detector 35 is used to monitor maternal heart rate multiple receivers will be disposed on light transmitter/detector 35. These receivers detect light reflected of different anatomical structures of the maternal patient. For example, one receiver detects light reflected off the maternal patient's arterial vessels to perform pulse oximetry as is known in the art. The other receiver detects light reflected off the uterine muscles of the maternal patient. When the light reflected off the uterine muscles of the maternal patient drops to significantly lower than the average light reflected off the uterus this indicates that the uterus is no longer contracted, since contracted muscles reflect less light than non-contracted muscles.

Also included within first housing 26 is accelerometer 52. Accelerometer 52 may be included within first housing 26 or may be external to first housing 26, for example as part of a wearable maternal heart rate monitor 50. Accelerometer 52 is disposed to detect movement of the maternal patient. Accelerometer 52 is communicatively connected to controller 38. Controller 38 utilizes the accelerometer data to determine when there has been movement of the maternal patient. Controller 38 utilizes the information about the movement of the maternal patient to check for and remove artifacts in the fetal heart rate data, as is known in the art.

Also included within first housing 26 is a user interface 58. User interface 58 may be a display screen, a speaker, an LED light bulb, or any other type of interface which can generate an alert to a caregiver who is monitoring a maternal and fetal patient. When controller 38 detects a drop in fetal heart such as is commensurate with an indication of fetal distress an alert can be generated through user interface 58 to alert a caregiver of such a condition. Additionally, when, as described above, controller 38 detects a heartbeat coincidence between the maternal patient and the fetal patient an alert can be generated to alert a caregiver to reposition ultrasound transducer 28 to ensure controller 38 is monitoring the fetal heart rate and not the maternal heart rate.

Also included within first housing 26 is a transmitter/receiver 60. Transmitter/receiver 60 is configured to transmit raw or filtered physiological data from controller 38 to an external patient monitor via the external patient monitor's receiving device. As described above, the transmitter/receiver 60 may be any device known in the art for wirelessly transmitting data between two points. The external patient monitor may exemplarily display the physiological data for a caregiver's attention or may store the data electronically, such as in an electronic patient record.

Also connected to controller 38, although located outside first housing 26, is maternal heart rate monitor 50. Maternal heart rate monitor 50 will not be connected to controller 38 in the case where a second receiver is used in the light transmitter/detector 35 to monitor maternal heart rate. Maternal heart rate monitor 50 is exemplarily a wearable heart rate monitor such as a wrist worn heart rate monitor or a finger worn heart rate monitor. Maternal heart rate monitor 50 may exemplarily further comprise an accelerometer configured to detect maternal patient movement. Maternal heart rate monitor 50 is wirelessly communicatively connected to controller 38. The wireless communicative connection may be a medical body area network (MBAN), and/or may exemplarily use Wi-Fi, Bluetooth, or ZigBee communication protocols or other RF communication protocols as may be recognized by a person of ordinary skill in the art.

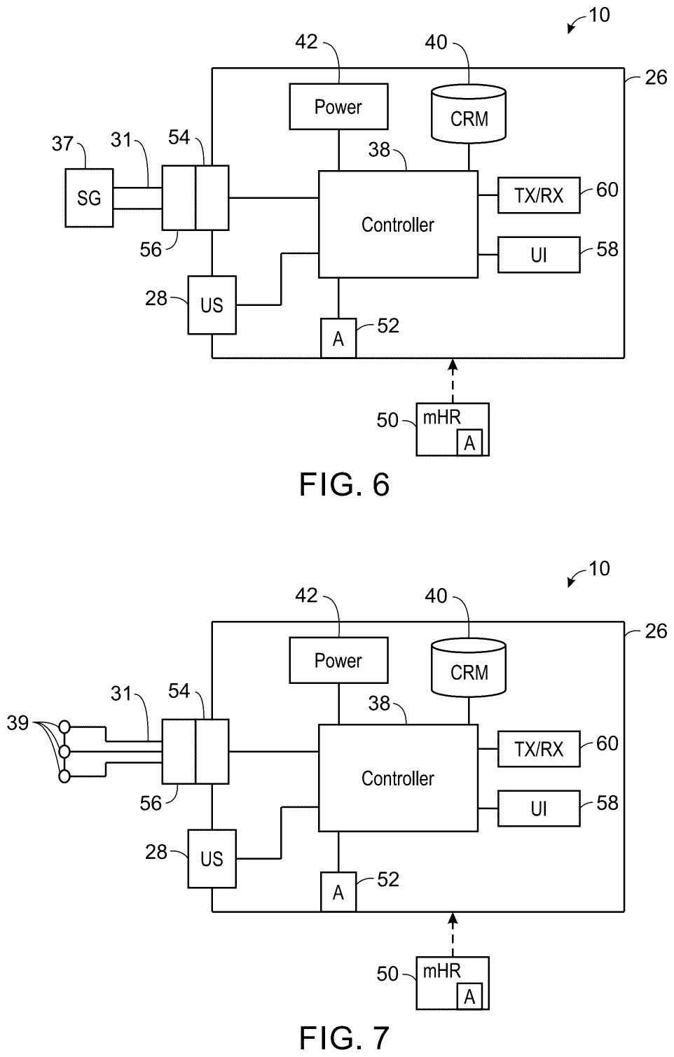

FIG. 6 is a schematic diagram of an exemplary embodiment of a maternal fetal monitor 10. Contained within the first housing 26 is an ultrasound transducer 28. The ultrasound transducer 28 exemplarily includes one or more ultrasound crystals. When the one or more ultrasound crystals receive a suitable excitation signal, the ultrasound crystals of the ultrasound transducer 28 produce an acoustic wave therefrom. In operation, the ultrasound crystals serve as both ultrasound transmitters and ultrasound receivers, and the ultrasound transducer 28 is operated to produce an acoustic wave form as described and then operated in a receive mode to receive the returned reflected acoustic signals back at the ultrasound transducer 28.

The ultrasound transducer 28 is connected to a controller 38. It will be recognized that controller 38 is exemplarily any of a variety of known controller circuits, integrated circuits, micro controllers, microprocessors, and associated circuitry. The controller 38 may exemplarily include a central processing unit (CPU) and integrated memory, although in embodiments the computer readable medium 40 comprising the memory may be a separate component or communicatively connected to the controller within the first housing 26. The controller exemplarily includes a processor that accesses software or firmware in the form of computer readable code stored on non-transient computer readable memory as either integrated memory or external memory. The processor executes the computer readable code as an instruction set to carry out the functions as described herein, including the receipt of input, calculations, and outputs as will be described. The first housing 26 further comprises a power source 42. The power source 42 is exemplarily a battery.

The maternal fetal monitor 10 further comprises a force gauge 37. Force gauge 37, such as a strain gauge, is disposed to be secured to the top half of a maternal patient's abdomen, near to the fundus of the uterus. Force gauge 37 is configured to be disposed on the maternal abdomen to detect and track the uterine activity of a maternal patient. Force gauge 37 may be exemplarily a MEMS-based strain gauge. In some embodiments, force gauge 37 may exemplarily be a patch-mounted strain gauge. Force gauge 37 is configured to detect strain in the maternal abdomen. Force gauge 37 directly measures electrical resistance. When the maternal abdomen is strained, for example due to a uterine contraction, the electrical resistance of force gauge 37 changes. By measuring the amount of change in electrical resistance of the gauge the force gauge 37 can also monitor the amount of strain on the maternal abdomen, which corresponds to the strength of the uterine contraction. In this way maternal fetal monitor 10 can measure not only the frequency and duration of uterine contractions but also their strength.

Also included within first housing 26 is accelerometer 52. Accelerometer 52 may be included within first housing 26 or may be external to first housing 26, for example as part of a wearable maternal heart rate monitor 50. Accelerometer 52 is disposed to detect maternal patient motion data. Accelerometer 52 is communicatively connected to controller 38. Controller 38 utilizes the maternal motion data to determine when there has been movement of the maternal patient. Controller 38 utilizes the information about the movement of the maternal patient to check for and remove artifacts in the fetal heart rate data and/or the from UA physiological measurements.

Also included within first housing 26 is a user interface 58. User interface 58 may be a display screen, a speaker, an LED light bulb, or any other type of interface which can generate an alert to a caregiver who is monitoring a maternal and fetal patient. When controller 38 detects a drop in fetal heart rate such as is commensurate with an indication of fetal distress an alert can be generated through user interface 58 to alert a caregiver of such a condition. Additionally, when, as described above, controller 38 detects a heartbeat coincidence between the maternal patient and the fetal patient an alert can be generated to alert a caregiver to reposition ultrasound transducer 28 to ensure controller 38 is picking up the fetal heart rate and not the maternal heart rate.

Also included within first housing 26 is a transmitter/receiver 60. Transmitter/receiver 60 is configured to transmit raw or filtered physiological data from controller 38 to an external patient monitor via the external patient monitor's receiving device. The transmitter/receiver 60 may be any device known in the art for wirelessly transmitting data between two points. In one embodiment, the transmitter/receiver 60 may be a body area network (BAN) device, such as a medical body area network (MBAN) device, that operates as part of a wireless network of wearable or portable computing devices. Other examples of radio protocols that could be used for this purpose are Bluetooth, Bluetooth Low Energy (BLE), ANT and ZigBee. The external patient monitor may exemplarily display the physiological data for a caregiver's attention or may store the data electronically, such as in an electronic patient record.

Also connected to controller 38, although located outside first housing 26, is maternal heart rate monitor 50. Maternal heart rate monitor 50 is exemplarily a wearable heart rate monitor such as a wrist-worn heart rate monitor or a finger-worn heart rate monitor. Maternal heart rate monitor 50 may exemplarily further comprise an accelerometer configured to detect maternal patient motion data. Maternal heart rate monitor 50 is wirelessly communicatively connected to controller 38. The wireless communicative connection may be via a medical body area network (MBAN), and/or may exemplarily use Wi-Fi, Bluetooth, or ZigBee communication protocols or other RF communication protocols as may be recognized by a person of ordinary skill in the art.

FIG. 7 is a schematic diagram of an exemplary embodiment of a maternal fetal monitor 10. Contained within the first housing 26 is an ultrasound transducer 28. The ultrasound transducer 28 exemplarily includes one or more ultrasound crystals. When the one or more ultrasound crystals receive a suitable excitation signal, the ultrasound crystals of the ultrasound transducer 28 produce an acoustic wave therefrom. In operation, the ultrasound crystals serve as both ultrasound transmitters and ultrasound receivers, and the ultrasound transducer 28 is operated to produce an acoustic wave form as described and then operated in a receive mode to receive the returned reflected acoustic signals back at the ultrasound transducer 28.

The ultrasound transducer 28 is connected to a controller 38. It will be recognized that controller 38 is exemplarily any of a variety of known controller circuits, integrated circuits, micro controllers, microprocessors, and associated circuitry. The controller 38 may exemplarily include a central processing unit (CPU) and integrated memory, although in embodiments the computer readable medium 40 comprising the memory may be a separate component or communicatively connected to the controller within the first housing 26. The controller exemplarily includes a processor that accesses software or firmware in the form of computer readable code stored on non-transient computer readable memory as either integrated memory or external memory. The processor executes the computer readable code as an instruction set to carry out the functions as described herein, including the receipt of input, calculations, and outputs as will be described. The first housing 26 further comprises a power source 42. The power source 42 is exemplarily a battery.

The maternal fetal monitor 10 further comprises EMG electrodes 39. EMG electrodes 39 collect biopotentials from the skin of the maternal patient. FIG. 7 depicts three electrodes 39 however it will be recognized that more or fewer electrodes 39 may be used in other embodiments. Electrodes 39 are configured to interface with the skin of a maternal patient and thereby acquire electrical biopotentials therefrom. In an example, while two electrodes may be sufficient to obtain a measurement of uterine activity, to obtain other measurement values, additional electrodes may be used. For example, three electrodes may be used when acquiring maternal heart rate measurements from biopotentials. In a non-limiting exemplary embodiment, the electrodes 39 may be constructed from silver and/or silver chloride, which may be formed as a foil or a conductive ink for construction of printed electronics. Additionally, at least one of the electrodes 39 may include a plurality of biocompatible conductive needles wherein each needle has a length between 10 micrometers and 200 micrometers. Such needles have been found to assist in providing an improved electric connection, for example by providing micro abrasion of the patient's skin through the stratum corneum to more conductive layers of skin below.

EMG electrodes 39 may be utilized to measure the impedance of a maternal patient's skin. When the impedance decreases this indicates that the uterus has contracted, as electrons move more easily through contracted muscles than through relaxed muscles. The impedance measurement may be used to synchronize detection of the fetal heart rate with detection of uterine activity. Additionally, the impedance may be used for artefact elimination, as impedance changes with maternal patient movement.

Also included within first housing 26 is accelerometer 52. Accelerometer 52 may be included within first housing 26 or may be external to first housing 26, for example as part of a wearable maternal heart rate monitor 50. Accelerometer 52 is disposed to detect maternal patient motion data. Accelerometer 52 is communicatively connected to controller 38. Controller 38 utilizes the maternal motion data to determine when there has been movement of the maternal patient. Controller 38 utilizes the information about the movement of the maternal patient to check for and remove artifacts in the fetal heart rate data and/or the from UA physiological measurements.

Also included within first housing 26 is a user interface 58. User interface 58 may be a display screen, a speaker, an LED light bulb, or any other type of interface which can generate an alert to a caregiver who is monitoring a maternal and fetal patient. When controller 38 detects a drop in fetal heart rate such as is commensurate with an indication of fetal distress an alert can be generated through user interface 58 to alert a caregiver of such a condition. Additionally, when, as described above, controller 38 detects a heartbeat coincidence between the maternal patient and the fetal patient an alert can be generated to alert a caregiver to reposition ultrasound transducer 28 to ensure controller 38 is picking up the fetal heart rate and not the maternal heart rate.

Also included within first housing 26 is a transmitter/receiver 60. Transmitter/receiver 60 is configured to transmit raw or filtered physiological data from controller 38 to an external patient monitor via the external patient monitor's receiving device. The transmitter/receiver 60 may be any device known in the art for wirelessly transmitting data between two points. In one embodiment, the transmitter/receiver 60 may be a body area network (BAN) device, such as a medical body area network (MBAN) device, that operates as part of a wireless network of wearable or portable computing devices. Other examples of radio protocols that could be used for this purpose are Bluetooth, Bluetooth Low Energy (BLE), ANT and ZigBee. The external patient monitor may exemplarily display the physiological data for a caregiver's attention or may store the data electronically, such as in an electronic patient record.

Also connected to controller 38, although located outside first housing 26, is maternal heart rate monitor 50. Maternal heart rate monitor 50 will not be connected to controller 38 in the case where a third electrode is used in electrodes 39 to monitor maternal heart rate. Maternal heart rate monitor 50 is exemplarily a wearable heart rate monitor such as a wrist worn heart rate monitor or a finger worn heart rate monitor. Maternal heart rate monitor 50 may exemplarily further comprise an accelerometer configured to detect maternal patient motion data. Maternal heart rate monitor 50 is wirelessly communicatively connected to controller 38. The wireless communicative connection may via be a medical body area network (MBAN), and/or may exemplarily use Wi-Fi, Bluetooth, or ZigBee communication protocols or other RF communication protocols as may be recognized by a person of ordinary skill in the art.

FIG. 8 is a diagram that shows an exemplary embodiment of the maternal and fetal monitoring system's power control. For example, the patch and the ultrasound sensing devices may be operated in an alternating pattern by controller 38 to level out the total power usage across the system 10 and minimize peak power requirements. Alternating the measurement operations between the fetal ultrasound sensor and the UA sensor—i.e., alternatively powering the ultrasound transducer 28 and the maternal measurement patch 29—levels out the power consumption of the maternal fetal monitor 10 to a constant or semi-constant level and the maximum power draw required to operating the sensing system. Moreover, alternating the measurement period reduces opportunities for interference between the sensing devices, as is described above.

As can be seen in FIG. 8, when current delivered to the first ultrasound transducer 28, represented by line 103, is high, there is no current flowing to the maternal measurement patch. Alternatively, when the current, represented by line 105, is being delivered to the maternal measurement patch, which may comprise any one of or any combination of an ultrasound sensor, a light transmitter/detector, a force measurement sensor, or multiple EMG patches, the first ultrasound transducer 28 is not operated and thus does not draw current. In this way power consumption by the maternal fetal monitoring system 10 is distributed over time and the peak current requirements of the system 10 are reduced. In addition, power is conserved by not powering two items at once, and the risk of noise detection is decreased.

FIG. 9 shows an exemplary embodiment of maternal measurement patch 29. Maternal measurement patch 29 may be configured to be disposably attached to the abdomen of a maternal patient, preferably near to the fundus of the maternal patient's abdomen, or otherwise above the maternal patient's umbilicus. In one embodiment, the maternal measurement patch 29 utilizes biocompatible adhesive to attach to the abdomen of a maternal patient. Maternal measurement patch 29 may comprise a printed circuit, or at least partially printed. Maternal measurement patch 29 may be configured to be a single-use disposable device. As described above, the maternal measurement patch 29 includes a flexible cable 31 that is configured to removably connect to the first housing. To that end, the maternal measurement patch 29 includes a first connection end 56 configured to removably connect to a connection port 54 on the first housing.

FIGS. 10A and 10B show an exemplary embodiment of connection end 56 and connection port 54. In some embodiments, connection end 56 and connection port 54 may be male/female respectively. In other embodiments, connection end 56 and connection port 54 may be female/male respectively. Connection end 56 and connection port 54 are configured to transmit data or analog signals from maternal measurement patch 29 via flexible cable 31 to first housing 28 and eventually to the controller 38. In some embodiments, the connection end 56 and connection port 54 are configured to transmit power from the first housing, such as from the battery or other power source 42, to any powered devices on the measurement patch (e.g., for the ultrasound, light transmission measurement, force gauge embodiments, and electrode embodiments where impedance measurements are performed between electrodes).

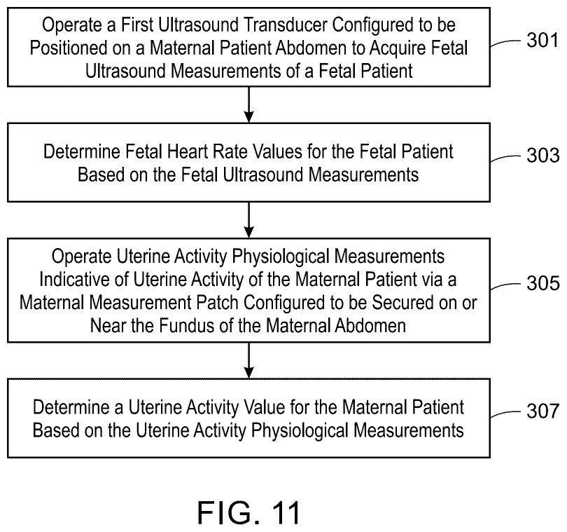

FIG. 11 shows an exemplary embodiment of a method of maternal and fetal monitoring. In step 301, a first ultrasound transducer configured to be positioned on a maternal patient abdomen is operated to acquire fetal ultrasound measurements of a fetal patient. The first ultrasound transducer may exemplarily be positioned on a maternal patient abdomen at or below the level of the umbilicus, near to the location of the fetus. At step 303 fetal heart rate values are determined for a fetal patient based on the fetal ultrasound measurements. Fetal heart rate values are measured using the Doppler effect, as is known in the art. As ultrasonic waves are reflected off the beating heart of the fetus a Doppler shift is observed which corresponds to the mechanical motion of the beating heart of the fetus. This Doppler shift can be used to calculate a fetal heart rate (fHR). At step 305 uterine activity physiological measurements indicative of uterine activity are obtained via a maternal measurement patch configured to be secured on or near the fundus of the maternal abdomen. At step 307 a uterine activity value for the maternal patient is determined based on the uterine activity physiological measurements.

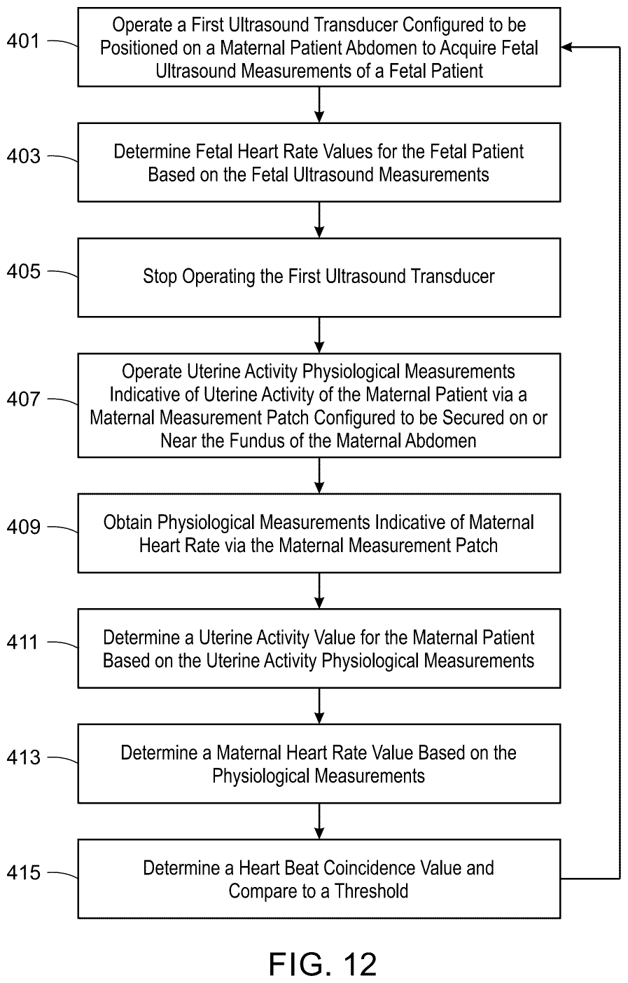

FIG. 12 shows an exemplary embodiment of a method of maternal and fetal monitoring. In step 401, a first ultrasound transducer configured to be positioned on a maternal patient abdomen is operated to acquire fetal ultrasound measurements of a fetal patient. The first ultrasound transducer may exemplarily be positioned on a maternal patient abdomen at or below the level of the umbilicus, near to the location of the fetus. At step 403 fetal heart rate values are determined for a fetal patient based on the fetal ultrasound measurements. Fetal heart rate values are measured using the Doppler effect, as is known in the art. As ultrasonic waves are reflected off the beating heart of the fetus a Doppler shift is observed which corresponds to the mechanical motion of the beating heart of the fetus. This Doppler shift can be used to calculate a fetal heart rate (fHR). At step 405 the first ultrasound transducer's operation is stopped and then the system is operated to obtain uterine activity physiological measurements via the patch at step 407. Ceasing operating the first ultrasound transducer while operating the maternal measurement patch to conduct UA and/or mHR measurements may reduce peak power demand across the system by operating only one sensing system at a time. Additionally or alternatively, alternating operations of the measurement systems may avoid interference therebetween, as is described above. At 407 uterine activity physiological measurements indicative of uterine activity of a maternal patient via a maternal measurement patch are obtained. A maternal measurement patch is configured to be disposably attached to the maternal patient abdomen, in some embodiments attached at or near to the fundus of the uterus. Uterine activity physiological measurements indicative of uterine activity of the maternal patient may include ultrasound measurements, light transmission measurements, force measurements, electrical potentials, or any combination thereof. At step 409 physiological measurements indicative of maternal heart rate are obtained via the maternal measurement patch. At step 411 a uterine activity value for the maternal patient based on the uterine activity physiological measurements is determined. At step 413 a maternal heart rate value based on the physiological measurements is determined. At step 416 heartbeat coincidence is assessed to determine whether coincidence is indicated.

The above-described steps of the processes of FIGS. 11 and 12 are not limited to the order and sequence shown and described in the figures and in various embodiments the steps may be performed in another order. Some of the above steps of the processes of FIG. 11 or 12 can be executed or performed substantially simultaneously where appropriate or in parallel to reduce latency and processing times.

This written description uses examples to disclose the invention(s), including the best mode, and also to enable any person skilled in the art to make and use the invention(s). Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention(s) is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

1. A maternal and fetal monitoring system comprising:
a first ultrasound transducer configured to be positioned on a maternal patient abdomen to acquire fetal ultrasound measurements of a fetus, the first ultrasound transducer housed in a first housing;
a maternal measurement patch configured to be secured on the maternal abdomen and to obtain UA physiological measurements indicative of uterine activity (UA) of the maternal patient;
a connection cable connecting the maternal measurement patch to the first housing and configured to transmit the UA physiological measurements from the maternal measurement patch to the housing of the first ultrasound transducer such that the UA physiological measurements are received by a controller; and
the controller configured to determine fetal heart rate (fHR) values for the fetus based on the fetal ultrasound measurements and to determine UA values for the maternal patient based on the UA physiological measurements.

2. The system of claim 1, wherein the maternal measurement patch is configured to be secured on an upper portion of the maternal abdomen.

3. The system of claim 2, wherein the connection cable is configured to enable placement of the first ultrasound transducer level with or below the umbilicus while the maternal measurement patch is secured above the umbilicus.

4. The system of claim 1, wherein the maternal measurement patch includes an adhesive and is configured to be secured to the maternal abdomen by adhering thereto.

5. The system of claim 1, wherein the connection cable includes a connection end configured to removably connect to a connection port on the first housing.

6. The system of claim 1, wherein the UA physiological measurements indicative of UA of the maternal patient include ultrasound measurements, light transmission measurements, force measurements, electrical potentials, or any combination thereof.

7. The system of claim 1, wherein the maternal measurement patch includes a second ultrasound transducer configured to acquire UA ultrasound measurements indicative of the UA of the maternal patient, wherein the controller is configured to determine the UA values based on the UA ultrasound measurements and wherein the second ultrasound transducer emits and measures a different ultrasound frequency than the first ultrasound transducer.

8. The system of claim 1, wherein the maternal measurement patch includes a second ultrasound transducer configured to acquire UA ultrasound measurements indicative of the UA of the maternal patient, wherein the controller is configured to determine the UA values based on the UA ultrasound measurements and wherein the controller is configured to control operation of the second ultrasound transducer to acquire the UA ultrasound measurements when the first ultrasound transducer is not operating to acquire the fetal ultrasound measurements.

9. The system of claim 1, wherein the maternal measurement patch includes a light transmission measurement device configured to acquire light transmission measurements indicative of the UA of the maternal patient, wherein the controller is configured to determine the UA values based on the light transmission measurements.

10. The system of claim 9, wherein the maternal measurement patch is further configured to acquire light transmission measurements indicative of maternal heart rate (mHR) values, and wherein the controller is further configured to determine a heartbeat coincidence based on the fHR values and the mHR values.

11. The system of claim 1, wherein the maternal measurement patch includes at least one of a force gauge sensor configured to acquire force measurements indicative of the UA of the maternal patient or a plurality of EMG sensors configured to acquire electrical potentials indicative of the UA of the maternal patient.

12. The system of claim 1, wherein the controller is housed in the first housing and further comprising a wireless transmitter in the first housing and configured to transmit at least the UA values and the fHR value to a receiving device.

13. The system of claim 1, further comprising a wireless transmitter in the first housing and configured to transmit the fetal ultrasound measurements and the UA physiological measurements to an external patient monitor, wherein the controller is in the external patient monitor.

14. The system of claim 1, wherein the maternal measurement patch is further configured to acquire measurements indicative of maternal heart rate (mHR) values, and wherein the controller is further configured to determine a heartbeat coincidence based on the fHR values and the mHR values.

15. The system of claim 1, wherein the controller is configured to receive maternal heart rate (mHR) values from a maternal heart rate monitor configured to be worn by the maternal patient and configured to transmit the mHR values, wherein the controller is further configured to determine a heartbeat coincidence based on the fHR values and the mHR values.

16. The system of claim 15, wherein the maternal heart rate monitor is a wrist-worn heart rate monitor configured to be worn on the maternal patient's wrist.

17. The system of claim 1, further comprising an accelerometer configured to measure motion of the maternal patient, wherein the controller is further configured to receive maternal motion data from the accelerometer and to use the maternal motion data to remove artifact from at least one of the UA physiological measurements and the fHR values.

18. A method of maternal and fetal monitoring, the method comprising:
operating a first ultrasound transducer configured to be positioned on a maternal patient abdomen to acquire fetal ultrasound measurements of a fetus;
determining fetal heart rate (fHR) values for the fetus based on the fetal ultrasound measurements;
when the first ultrasound transducer is not operating to acquire the fetal ultrasound measurements, obtaining UA physiological measurements indicative of uterine activity (UA) of the maternal patient via a maternal measurement patch configured to be secured on an upper portion of the maternal abdomen; and
determining UA values for the maternal patient based on the UA physiological measurements.

19. The method of claim 18, wherein the UA physiological measurements indicative of UA of the maternal patient include ultrasound measurements, light transmission measurements, force measurements, electrical potentials, or any combination thereof.

20. The method of claim 18, when the first ultrasound transducer is not operating to acquire the fetal ultrasound measurements, obtaining physiological measurements indicative of maternal heart rate (mHR) via the maternal measurement patch, and wherein the controller is further configured to determine mHR values based on the physiological measurements and to determine a heartbeat coincidence based on the fHR values and the mHR values.
